(19) United States
(12) Patent Application Publication
(10) Pub. No.: US 2026/0162339 A1
(43) Pub. Date: Jun. 11, 2026

(54) MEDICAL IMAGE ARTIFACT MITIGATION DURING AN IMAGING EXAMINATION BASED ON AN ASSESSMENT OF RAW IMAGE DATA FROM THE IMAGING SYSTEM

(71) Applicant: GE Precision Healthcare LLC, Waukesha, WI (US)

(72) Inventors: Subhashis Banerjee, KOLKATA (IN); Sudhanya Chatterjee, Bengaluru (IN); Dattesh Dayanand Shanbhag, Bengaluru (IN)

(73) Assignee: GE Precision Healthcare LLC, Waukesha, WI (US)

(21) Appl. No.: 18/973,318

(22) Filed: Dec. 9, 2024

(51) Int. Cl. G06T 11/00; G06T 7/00; G16H 30/20; G16H 30/40

(52) U.S. Cl. CPC G06T 12/30; G06T 7/0012; G16H 30/20; G16H 30/40; G06T 2207/30016; G06T 2207/30168

(57) ABSTRACT

A computer-implemented method includes obtaining raw image data from a medical imaging system during an imaging examination performed by the medical imaging system, processing the raw image data with trained artificial intelligence deep learning based artifact models to detect artifact from the raw medical image data and generate multi-dimensional information about the detected artifact, processing at least the multi-dimensional information with trained large vision-language models to generate a summary of the detected artifact and determine mitigation for the artifact, and presenting the summary and mitigation. The summary includes a set of images and indicates a type of each detected artifact, an identification of images in the set of images that includes the detected artifact, and a severity of the detected artifact, and the mitigation provides acts for reducing the image artifact for the remainder of the imaging examination. A least a portion of the mitigation is implemented.

### FIELD

The following generally relates to medical imaging, and more particularly to medical image artifact mitigation during an imaging examination with an imaging system based on an assessment of raw image data from the imaging system, and is amenable to non-medical image artifact mitigation.

### BACKGROUND

Medical imaging systems such as Magnetic Resonance (MR), Computed Tomography (CT), Positron Emission Tomography (PET), Single Photon Emission Tomography (SPECT), etc. scanners generate raw data (e.g., k-space data for an MR scan, sinogram data for a CT scan, a PET scan and a SPECT scan, etc.) indicative of an interior of a subject, which is reconstructed to generate volumetric image data of the interior of the subject or object. The volumetric image data can be variously manipulated to generate a set of image slices that can be displayed on a two-dimensional (2-D) display monitor and/or saved. Different manufacturers of such scanners utilize different formats for their data. As such, the data of one manufacturer may not be readily viewable by another manufacturer and/or other device.

To share image, the set of image slices have been converted to the Digital Imaging and Communications in Medicine (DICOM) format, which is an industry “standard” for transmitting, viewing, and storing medical images. For example, after an imaging examination a set of image slices have been encoded in the DICOM format and transmitted to a radiology viewing station, such as a Picture Archiving and Communication System (PACS), for evaluation (e.g., reading and interpreting) by a radiologist. The evaluation, which may occur a few days after the imaging examination, includes reviewing the DICOM formatted images for a presence of a finding such as an abnormality, a change in a known abnormality, etc. The raw image data generated by the imaging system includes artifact that may be visible in the DICOM formatted images.

The artifact, which can be patient, image acquisition and/or imaging system related, has appeared as an abnormality and/or obscured an abnormality. Where a radiologist determines the artifact affects accurate interpretation such that the DICOM formatted images are not diagnostic quality, the radiologist orders a rescan. A rescan requires scheduling the patient to return to the imaging department at a later date for another imaging examination. Unfortunately, a rescan of the patient increases cost and consumes time, for both the patient and the imaging department, and reduces overall throughput of the imaging department. In addition, a rescan does not guarantee that the new DICOM formatted images will not include similar artifact and/or be diagnostic quality, and another rescan may need to be scheduled.

In view of the foregoing, there is an unresolved need for an improved approach that at least mitigates the above-noted and/or other shortcomings of the existing technology and/or a technological field.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a computer-implemented method includes obtaining raw image data from a medical imaging system during an imaging examination performed by the medical imaging system. The computer-implemented method further includes processing the raw image data with trained artificial intelligence deep learning based artifact models to detect artifact from the raw medical image data and generate multi-dimensional information about the detected artifact. The computer-implemented method further includes processing at least the multi-dimensional information with trained large vision-language models to generate a summary of the detected artifact and determine mitigation for the artifact. The computer-implemented method further includes presenting the summary and mitigation. The summary includes a set of images and indicates a type of each detected artifact, an identification of images in the set of images that includes the detected artifact, and a severity of the detected artifact, and the mitigation provides acts for reducing the image artifact for the remainder of the imaging examination. A least a portion of the mitigation is implemented.

In another aspect, a system includes a memory and a processor. The memory includes an artifact mitigation module configured to assess raw medical image data for artifact and provide a summary of the assessment and mitigation to reduce the artifact. The artifact mitigation module includes deep learning based artifact models trained to assess raw image data, detect artifact in the raw medical image data, and generate multi-dimensional information about the detected artifact and large vision-language models trained to process the multi-dimensional information and generate a summary of the detected artifact and mitigation for the artifact. The processor is configured to execute the trained deep learning based artifact models to assess raw image data received from an imaging system, detect artifact in the raw medical image data, and generate multi-dimensional information about the detected artifact. The processor is configured to execute the trained large vision-language models trained to process the multi-dimensional information and generate a summary of the detected artifact and mitigation for the artifact, and a display configured to present the summary of the detected artifact and the mitigation for the artifact.

In another aspect, a computer readable medium is encoded with computer executable instructions. The computer executable instructions, when executed by a processor, cause the processor to obtain raw image data from a medical imaging system during an imaging examination performed by the medical imaging system, process the raw image data with trained artificial intelligence deep learning based artifact models to detect artifact from the raw medical image data and generate multi-dimensional information about the detected artifact, process the multi-dimensional information with train large vision-language models to generate a summary of the detected artifact and determine mitigation for the artifact, and present the summary and mitigation The summary includes a set of images and indicates a type of each detected artifact, an identification of images in the set of images that includes the detected artifact, and a severity of the detected artifact, and the mitigation provides acts for reducing the image artifact for the remainder of the imaging examination. A least a portion of the mitigation is implemented.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates an example of an imaging system that includes an artifact mitigation module, in accordance with an embodiment(s) herein.

FIG. 2 schematically illustrates an example of the artifact mitigation module, in accordance with an embodiment(s) herein.

FIG. 3 schematically illustrates an example of generating a training set for the artifact mitigation module, in accordance with an embodiment(s) herein.

FIG. 4 schematically illustrates an example of training the artifact mitigation module, in accordance with an embodiment(s) herein.

FIG. 5 schematically illustrates an example of employing the trained artifact mitigation module to assess raw image data, in accordance with an embodiment(s) herein.

FIG. 6 schematically illustrates an example of generating an MR image data training set for the artifact mitigation module, in accordance with an embodiment(s) herein.

FIG. 7 schematically illustrates an example of the MR training data, in accordance with an embodiment(s) herein.

FIG. 8 schematically illustrates an example of training the artifact mitigation module with the MR image data, in accordance with an embodiment(s) herein.

FIG. 9 schematically illustrates an example of employing the trained artifact mitigation module to assess raw MR image data, in accordance with an embodiment(s) herein.

FIG. 10 schematically illustrates an example of a computing system that includes the artifact mitigation module, in accordance with an embodiment(s) herein.

FIG. 11 illustrates a non-limiting example of a flow chart for a computer-implemented method for assessing raw medical image data for artifact and mitigating the artifact with a trained artifact mitigation module, in accordance with an embodiment(s) herein.

FIG. 12 illustrates a non-limiting example of a flow chart for a computer-implemented method for assessing raw MR image data for artifact and mitigating the artifact with a trained artifact mitigation module, in accordance with an embodiment(s) herein.

### DETAILED DESCRIPTION

Embodiments of the present disclosure will now be described, by way of example, with reference to the figures, in which a system, a method and/or computer executable instructions on a computer readable medium provide real-time (i.e., before the patient examination is complete) artificial intelligence (AI)-based medical image artifact mitigation through an image quality assessment of raw image data (which includes data generated by the image system, e.g., k-space and reconstructed image data for MR, sinogram and reconstructed image data for CT, PET and SPECT, etc., but not further processed such as encoded in the DICOM and/or other format) during the patient examination, including a presentation of results of the assessment and mitigation. The presentation includes information for improving image quality prior to completion of the imaging examination.

As discussed above, with existing technology, after an imaging examination has been completed, images are encoded in the DICOM format and conveyed to radiology viewing station such as a PACS for subsequent evaluation by a radiologist, e.g., for a presence of a finding, a change in a known finding, etc. Where the radiologist determines that image artifact negatively affects accurate interpretation of the DICOM images, e.g., obscuring details, etc., the radiologist orders another imaging examination of the patient for a rescan to acquire another set of DICOM images, with an expectation that the image quality of a new set of DICOM images will allow for accurate interpretation, although another rescan may need to be scheduled. Again, a rescan requires the patient to schedule another examination, which increases cost and consumes time for the patient and the imaging department.

As described in greater detail below, the AI based approach herein includes generating training data (also referred to herein as synthetic artifact induced raw image data) by adding simulated artifact to artifact-free raw data (e.g., cases previously reviewed and labeled by a radiologist) in a manner that accurately emulates artifact manifestation in the raw image data. The training data is employed to train multi-dimensional AI models, which are utilized to assess raw image data generated during an imaging examination of a patient. Where the trained AI models detect artifact in the raw image data under assessment, the trained AI models present information (such as a type of artifact, which image(s) includes the artifact, a severity of the artifact, a region(s) within an image with the artifact, etc.), along with mitigation to avoid and/or reduce the artifact. By performing the mitigation during the imaging examination, the approach described herein mitigates the added cost and time consumption of scheduling and performing another imaging examination.

The raw image data includes complex-valued data (e.g., magnitude and phase with k-space data, sinograms, etc.), unlike DICOM and/or other spatial domain formatted images that include real-valued magnitude images. Furthermore, certain artifacts are well-pronounced in the raw image data domain. The AI-based approach described herein processes raw image data in the raw image data domain, which allows for qualitative and/or quantitative assessment of the unique structure and complex-valued nature of the raw image data, providing an improvement relative to artifact detection approaches configured to detect artifacts in DICOM images in the spatial domain. The presentation of the results and mitigation assists an operator by providing image quality (IQ) feedback on the medical images acquired while the patient is still in the scanner, enabling informed decisions regarding scan parameter and/or adjustments before the imaging examination is completed.

As briefly discussed above, the artifact mitigation approach described herein can be employed at least with medical imaging systems such as MR, CT, PET, SPECT, etc., and also with non-medical imaging systems. For explanatory purposes, sake of brevity, and/or clarity, the artifact mitigation approach is described in an example in connection with an imaging system configured for MR imaging. In one instance, the imaging system is a dedicated MR imaging system. In another instance, the imaging system is a hybrid imaging system that includes MR and at least one other imaging modality such as PET, etc. However, it is to be understood that the mitigation approach can additionally, or alternatively, be employed with other imaging systems.

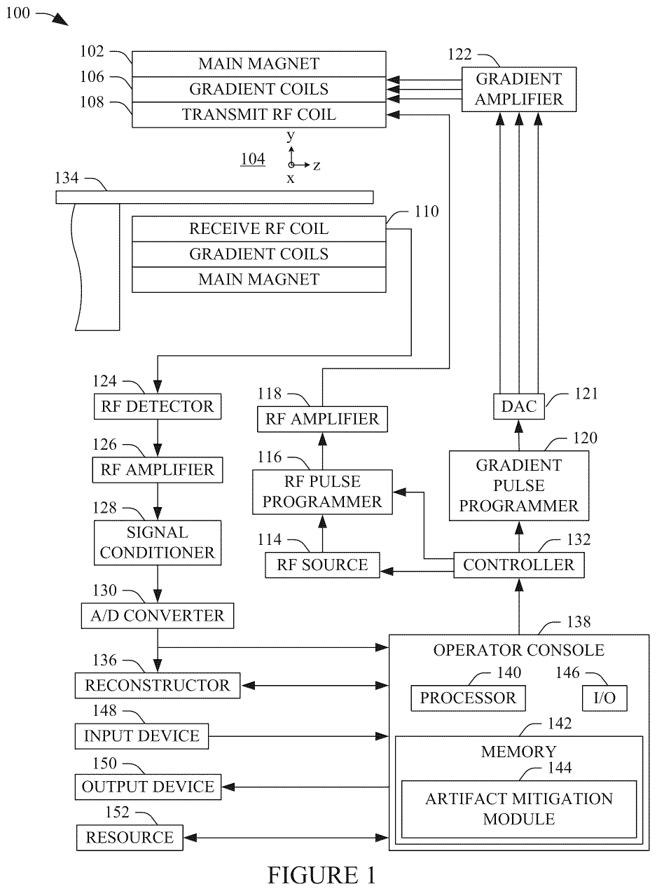

Initially referring to FIG. 1, an imaging system 100 configured at least for magnetic resonance (MR) imaging is schematically illustrated. The imaging system 100 includes a main magnet 102. The main magnet 102 is configured to provide a substantially homogeneous, temporally constant main magnetic field B₀ in an examination region 104. Depending on the desired main magnetic field strength and the requirements of a particular application, various magnet technologies (e.g., superconducting, resistive, or permanent magnet technologies) and/or physical magnet configurations (e.g., solenoidal or open magnet configurations) have been implemented.

The imaging system 100 further includes gradient coils 106. The gradient coils 106 are configured to generate time varying magnetic gradient fields. The gradient coils 106 include an x-gradient coil for generating a gradient field along the x-direction, a y-gradient coil for generating a gradient field along the y-direction and a z-gradient coil for generating a gradient field along the z-direction. A function of the gradient coils 106 is to spatially encode the MR signal to differentiate signals from different locations within the body. The gradient coils 106 are also utilized for various techniques like diffusion imaging, perfusion imaging, functional imaging, elastography imaging, angiography imaging, etc. For diffusion imaging, the gradient coils 106 are configured to generate diffusion-sensitizing gradients that affect the image contrast.

The imaging system 100 further includes a transmit radiofrequency (RF) coil 108. The transmit RF coil 108 is configured to generate RF signals that excite and/or otherwise manipulate hydrogen and/or other magnetic resonant active nuclei in an object and/or subject in the examination region 104. The imaging system 100 further includes a receive RF coil 110. The receive RF coil 110 is configured to receive magnetic resonance (MR) signals generated by the excited nuclei in the examination region 104. The illustrated transmit RF coil 108 and receive RF coil 110 are volume or whole-body coils integrated in the imaging system 100.

In another example, the RF coil 108 is configured as the receive coil, and the RF coil 110 is configured as the transmit coil. In another instance, the transmit RF coil 108 and receive RF coil 110 are part of a same transmit-receive RF coil and a switch or the like is configured to switch between transmit and receive operations. In another instance, the transmit RF coil 108 and receive RF coil 110 are separate from the imaging system 100 and are installed in the imaging system 100 for use therewith to scan the object or subject. Other coils are contemplated herein. Examples include smaller volume coils configured for extremities such as the head, etc., surface coils, etc.

The imaging system 100 further includes an RF source 114. The RF source 114 is configured to generate an RF signal having a desired frequency (e.g., the Larmor frequency of the MR active nuclei under investigation). The imaging system 100 further includes an RF pulse programmer 116. The RF pulse programmer 116 is configured to establish a timing and/or a shape of the RF signal generated by the RF source 114. The imaging system 100 further includes an RF amplifier 118. The RF amplifier 118 is configured to amplify the shaped RF signal to levels required by the transmit RF coil 108 for exciting nuclei in the object or subject in the examination region 104.

The imaging system 100 further includes a gradient pulse programmer 120. The gradient pulse programmer 120 is configured to establish a timing, a strength and/or a shape of the time varying magnetic fields that are generated by the gradient coils 106 during a scan of an object and/or subject. A digital-to-analog converter (DAC) 121 converts the digital output of the gradient pulse programmer 120 to analog signals for the respective gradient coils 106. The imaging system 100 further includes a gradient amplifier 122. The gradient amplifier 122 is configured to amplify the time varying magnetic fields to levels required by the respective gradient coils 106. The gradient amplifier 122 includes an independent power amplifier for each of the gradient coils 106, including the x-gradient coil, the y-gradient coil and the z-gradient coil. In one example, the x- and y-gradient coils respectively include a saddle (Golay) coil and the z-gradient coil includes a circular (Maxwell) coil.

A controller 132 controls the RF source 114, the RF pulse programmer 116 and the gradient pulse programmer 120. The RF pulse programmer 116 and the gradient pulse programmer 120 respectively control the RF amplifier 118 and the gradient amplifier 122 based on an imaging technique being employed for a scan of an object or subject. Examples of different imaging techniques include diffusion imaging, perfusion imaging, functional imaging, elastography imaging, angiography imaging, etc.

The imaging system 100 further includes an RF detector 124. The RF detector 124 is configured to receive an analog MR signal generated by the RF receive coil 110 during a data acquisition window having a given timing and length. The imaging system 100 further includes an RF amplifier 126. The RF amplifier 126 is configured to amplify the received analog MR signal. The imaging system 100 further includes a signal conditioner 128. The signal conditioner 128 is configured to condition the amplified analog MR signal, e.g., demodulate, filter, etc., the amplified MR signal. The imaging system 100 further includes an analog-to-digital (A/D) converter 130. The A/D converter 130 is configured to digitize the conditioned analog MR signal, i.e., convert the conditioned analog MR signal into a digital MR signal.

The imaging system 100 further includes a subject/object support 134. The subject/object support 134 includes a tabletop moveably coupled to a frame/base. In one instance, the tabletop is slidably coupled to the frame/base via a bearing or the like, and a drive system (not visible) including a controller, a motor, a lead screw, and a nut (or other drive system) translates the tabletop along the frame/base into and out of the examination region 104. The tabletop is configured to support an object or subject in the examination region 104 for loading, scanning, and/or unloading the subject or object. A table controller (not visible) controls the drive system.

The imaging system 100 further includes a reconstructor 136. The reconstructor 136 is configured to reconstruct the digitized MR signals and generate individual axial (2-D) images and/or volumetric (3-D) image data. The MR signals include encoded imaging data (i.e., k-space data), which is transformed by the image reconstruction algorithm using a Fourier transform and/or other algorithm to generate volumetric D image data. The volumetric image data can be variously manipulated to generate 2-D slices that can be visually presented via a display monitor, printed, etc. Saved images can be encoded in the DICOM format and transferred to another device such as a radiology viewing station such as a PACS or the like.

The imaging system 100 further includes a computing system 138 such as a computer, a workstation, etc. The computing system 138 serves as an “operator console” of the imaging system 100. The operator console 138 includes at least one processor 140 such as a microprocessor (UP), a central processing unit (CPU), graphics processing unit (GPU), etc., and a computer readable medium 142 (“MEMORY”), which includes non-transitory medium and excludes transitory medium (signals, carrier waves, and the like). The at least one processor 140 is configured to provide control signals to the controller 132 to control the RF source 114, the RF pulse programmer 116 and the gradient pulse programmer 120, e.g., for acquiring MR signals.

The computer readable medium 142 at least includes an artifact mitigation module 144. As described in greater detail below, the artifact mitigation module 144 is configured to provide AI-based medical image artifact mitigation through an image quality assessment of raw image data during the patient examination and provide results of the image quality assessment and mitigation. As discussed above, with existing technology, after an imaging examination has been completed, the image data is encoded in the DICOM format and conveyed to radiology viewing station such as a PACS for subsequent evaluation by a radiologist. Where the radiologist determines that image artifact negatively affects accurate interpretation of the DICOM images, the patient is scheduled to return for another imaging examination, which increases cost and consumes time for the patient and the imaging entity.

The AI-based approached described herein processes raw image data in the raw image data domain (e.g., magnitude and phase), which allows for qualitative and/or quantitative assessment of the raw image data. In one instance, this provides an improvement relative to a configuration in which the AI-based approach is configured to process real-valued image data and operate in the spatial domain, and not complex medical image data. The presentation of the results and mitigation assists the user by providing IQ feedback on the medical images acquired while the patient is still in the scanner, enabling informed decisions regarding scan parameter adjustments, etc. before the imaging examination is completed to provide medical images with an image quality that avoids having to schedule another imaging examination to redo the scan.

The imaging system 100 further includes input/output (I/O) 146. An input device 148 includes a keyboard, mouse, touchscreen, microphone, etc. An output device 150 includes a human readable device such as a display monitor or the like. A remote resource 152 includes one or more of a server, a workstation, a Radiology Information System (RIS), a Hospital Information System (HIS), an Electronic Medical Record (EMR), a PACS, one or more other MR scanners, cloud processing resources (which includes shared remote data storage and/or computing power, including processing resources distributed over multiple locations/data centers), etc. The input device 148, the output device 150, and/or the remote resource 152 are in communication with the computing system 138 through the I/O 146 and/or otherwise.

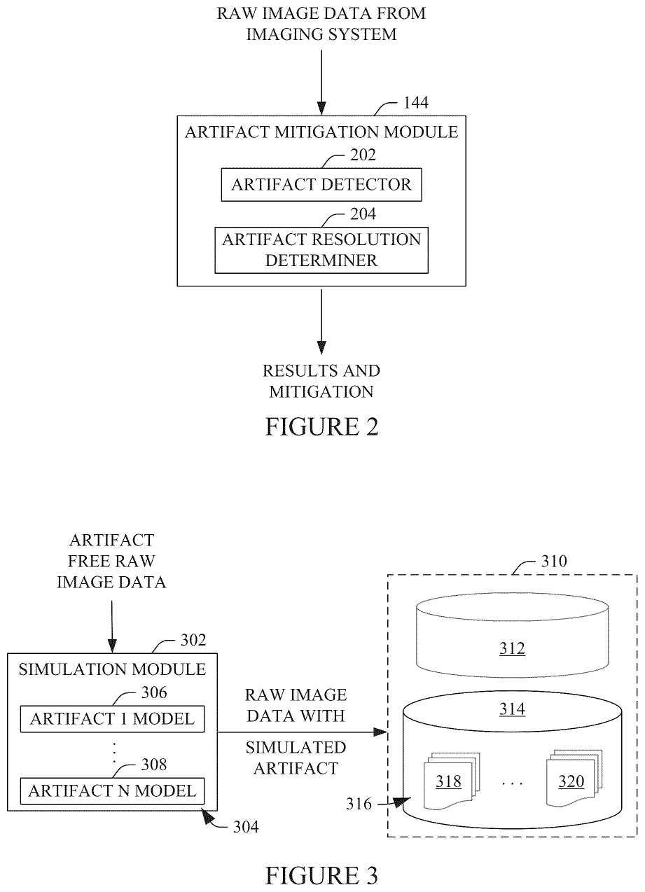

Turning to FIG. 2, a non-limiting example of the artifact mitigation module 144 is schematically illustrated. The example of the artifact mitigation module 144 includes an artifact detector 202 and an artifact resolution determiner 204.

The artifact detector 202 receives, as input, raw image data generated by an imaging system such as the imaging system 100 (FIG. 1) and/or other imaging system during an imaging examination of a patient. The artifact detector 202 includes AI models trained to detect artifact in complex-valued data sets of data such as the raw image data produced by the imaging system 100 to detect artifacts and provide multidimensional information for detected artifacts. For the training, the data includes at least synthetic artifact induced raw image data, which includes known artifact free raw image data with simulated artifact added thereto.

The artifact resolution determiner 204 receives, as input, detection results from the artifact detector 202 during the imaging examination of the patient. The artifact resolution determiner 204 includes AI models trained to extract certain information from the detection results and generate and present parts of the results along with mitigation to avoid and/or reduce the detected artifact during a remainder of the imaging examination. Examples of the certain information include, but are not limited to, type of artifact, severity of the artifact, etc. The operator of the imaging system reviews the results and mitigation and can implement at least part of the mitigation and/or other mitigation, or not mitigation.

The approach described herein provides varies benefits and/or improvements in a technology and/or technological field. For example, in one instance the approach described herein reduces cost and time, e.g., by reducing a number of scans, both the healthcare facility and/or the patient, which preserves time and/or resources. In addition, the approach described herein mitigates multiple appointments, improving the overall throughput and lowering operational cost. Furthermore, it reduces interobserver variability and establishes consistent quality criteria.

In addition, the approach described herein processes complex-valued raw image data in the raw image data domain (e.g., both magnitude and phase) during an imaging examination, which allows for qualitative and/or quantitative assessment of the raw image data before the end of the imaging examination, providing an improvement over a configuration that otherwise is configured to process real-valued image data in the spatial domain (e.g., magnitude only) after completion of the imaging examination, and allowing for detection of certain artifacts that are well-pronounced in the raw image data domain.

In another instance, the approach described herein further provides for consistency and/or accuracy, e.g., consistent, objective artifact detection, which reduces human error and/or variability, ensuring higher quality scans across different machines and operators. In another instance, the approach described herein further provides for streamlined scanning. For example, while one scan is being performed, the imaging technician can prepare for the next sequence of the imaging examination, with the system providing an alert and/or recommendation for artifacts appearing in a scan. In another instance, the approach described herein facilitates inexperienced operators who may not flag artifacts and/or adjust scan parameters to avoid image artifacts.

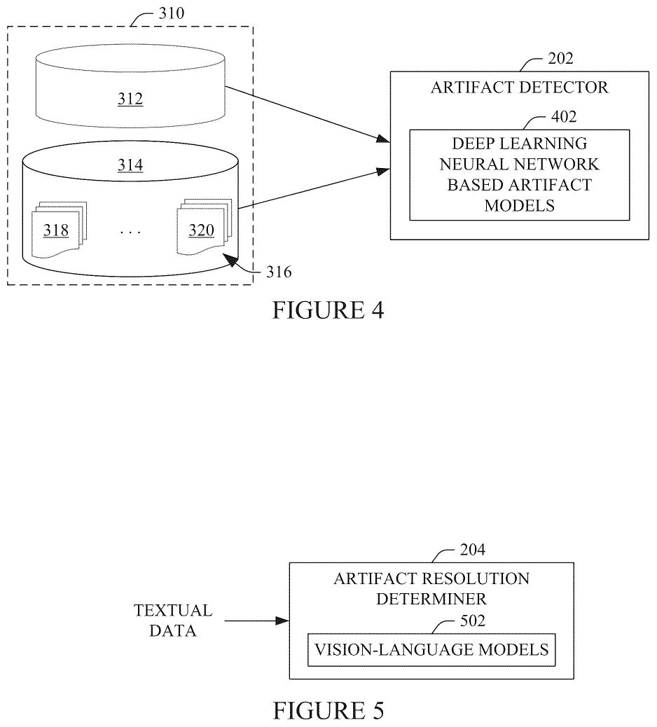

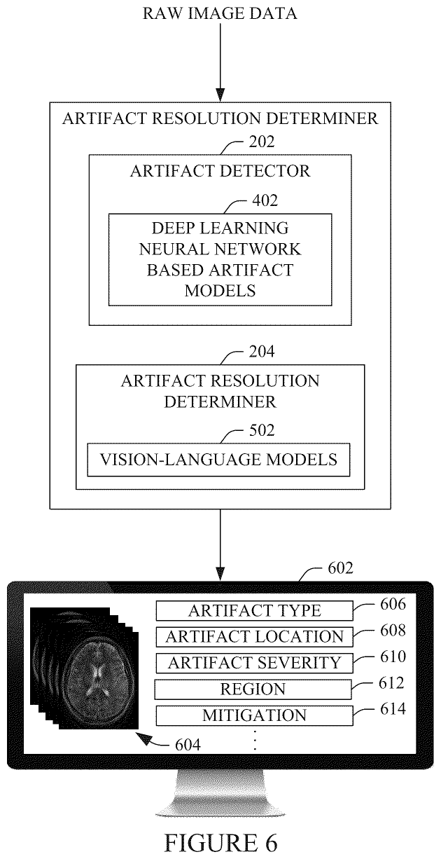

FIGS. 3, 4, 5 and 6 provide an example of training and using the artifact mitigation module 144. FIG. 3 schematically illustrates an example of generating training data to train the artifact detector 202 (FIG. 2). FIG. 4 schematically illustrates an example of training the artifact detector 202. FIG. 5 schematically illustrates an example of training the artifact resolution determiner 204. FIG. 6 schematically illustrates an example of utilizing the trained artifact detector 202 and the trained artifact resolution determiner 204 to assess raw image data from an imaging system and present results and mitigation.

Initially referring to FIG. 3, an example simulation module 302 is schematically illustrated. In one instance, the simulation module 302 is included in the memory 142 of the operator console 138 of the imaging system 100 (FIG. 1). Additionally, or alternatively, the simulation module 302 is included in a device of the resource 152 (FIG. 1). For example, in one instance the simulation module 302 is included in a computing device that is separate from the imaging system 100, part of cloud-based remote resources, distributed across a network, etc. The simulation module 302 is configured to receive, as input, artifact free raw image data.

As utilized herein, the term “artifact free” encompasses raw image data used to generate DICOM and/or other images that were assessed by a radiologist and determined to have no observable artifact or visible artifact under a predetermined observable threshold amount of artifact for tissue of interest as determined by the radiologist. The artifact information for the DICOM images can be determined via a file header, a DICOM annotation field, a corresponding radiology report, etc. The simulation module 302 is configured to process the raw image based on artifact models 304 and output synthetic artifact induced raw image data (i.e., the raw image data with the simulated artifact).

In the illustrated example, the artifact models 304 include N artifact models, including an artifact model 306 (“artifact 1 model”), . . . , and an artifact model 308 (“artifact N model”), where N is an integer greater than or equal to one. In this example, the artifact models 304 (the artifact model 306, . . . , and the artifact model 308) are configured to introduce artifacts in the raw image data in a physics informed manner that accurately emulates artifact manifestation, as if the artifact manifested during data acquisition. The simulations replicate artifacts in both the magnitude and phase data of the raw image data. Each of the artifact models 304 is configured for a corresponding artifact that is parameterized and simulated in a forward model. The simulation module 302 adds a simulated artifact and generates artifact labels for the artifact. Known and/or other approaches are contemplated herein.

A storage unit 310 includes a label storage region 312 for storing artifact labels and a raw image data storage region 314 for storing the synthetic artifact induced raw image data. In another instance, the storage region 312 and the storage region 314 are the same storage region (the storage region 312, the storage region 314, or another storage region). In yet another instance, at least one of the storage regions 312 and the storage region 314 is not part of the storage region 312, and is located in other storage. In one instance, the storage 312 is part of the memory 142 of the operator console 138 of the imaging system 100. In another instance, the storage region 312 is part of another device such as one or more devices of the resource 152 (FIG. 1) and/or other device.

In the illustrated example, N sets of synthetic artifact induced raw image data 316 are stored in the raw image data storage region 314, including a synthetic artifact induced raw image data set 318, . . . , and a synthetic artifact induced raw image data set 320. The N sets of synthetic artifact induced raw image data 316 correspond to the N artifact models 308. For example, in one instance, the synthetic artifact induced raw image data set 318 includes raw image data with artifact simulated using the artifact model 306, . . . , and the synthetic artifact induced raw image data set 318 includes raw image data with artifact simulated using the artifact model 308. Different raw image data and/or parameters of an artifact model are used to generate the different data in any set of the raw image data. For example, artifact can be applied across different contrast, anatomy, various clinical applications (including cardiac, head and spine, body and musculoskeletal), etc.

FIG. 4 schematically illustrates training the artifact detector 202 with the training data created in connection with FIG. 3 and/or otherwise. In this example, the artifact detector 202 includes neural network based artifact models such as deep learning neural network based artifact models 402, complex-valued deep learning neural network based artifact models, etc., and/or other AI models. In this example, the artifact detector 202 (e.g., the deep learning based artifact models 402) is trained with the N sets of synthetic artifact induced raw image data 316 (the synthetic artifact induced raw image data set 318, . . . , and the synthetic artifact induced raw image data set 320) and the corresponding artifact labels in the labels storage region 312. The artifact detector 202 is trained to detect multi-dimensional information about the artifact.

FIG. 5 schematically illustrates training the artifact resolution determiner 204. In this example, the artifact resolution determiner 204 includes large vision-language models (VLM) 502 and/or other AI models. In this example, the artifact resolution determiner 204 (e.g., the large vision-language models) is trained with image data and text-based data, enabling the artifact resolution determiner 204 to perform natural language processing (NLP) tasks such as correlations between imaging features and text information for text summarization, etc. In one instance, the large vision-language models are based on a transformer architecture that utilizes self-attention to focus on different parts of input text, allowing the large vison-language models to understand the context and relationships between words and imaging features.

FIG. 6 schematically illustrates an example of utilizing the trained artifact detector 202 and the artifact resolution determiner 204. The artifact detector 202 receives, as input, raw image data generated by an imaging system during an imaging examination of a patient. The artifact detector 202 processes the received raw image data with the complex-valued deep learning neural network and outputs results that includes multi-dimensional information, e.g., at least an artifact was detected, a type of the detected artifact, an image slice(s) that includes the artifact, a severity of the artifact, a region within a slice where the artifact it located, etc.

The large vision-language models 502 receive, as input, the results and imaging features from the artifact detector 202 during the imaging examination of the patient. The large vision-language models 502 evaluate the results and displays at least a portion of the multi-dimensional information and mitigation of the artifact. In this example, the summary of the results and mitigation are displayed via a display monitor 602. In one instance, the display monitor 602 is a display monitor of an output device of the imaging system. In another instance, the display monitor 602 is a display monitor separate from the imaging system, e.g., a separate computing system utilized in connection with the imaging system, etc., such as a third-party computing system or the like.

In this example, the summary includes a set of images 604. The summary further includes an artifact type 606 that includes a textual description of a type of a detected artifact. The summary further includes an artifact location 608 that includes a text description indicating which images of the set of images 604 includes the detected artifact. The summary further includes an artifact severity 610 that indicates a severity of the detected artifact. The summary further indicates a region 612 in the images where the artifact is detected. The summary further includes suggested acts of artifact mitigation 614. Other information can additionally, or alternatively, be presented.

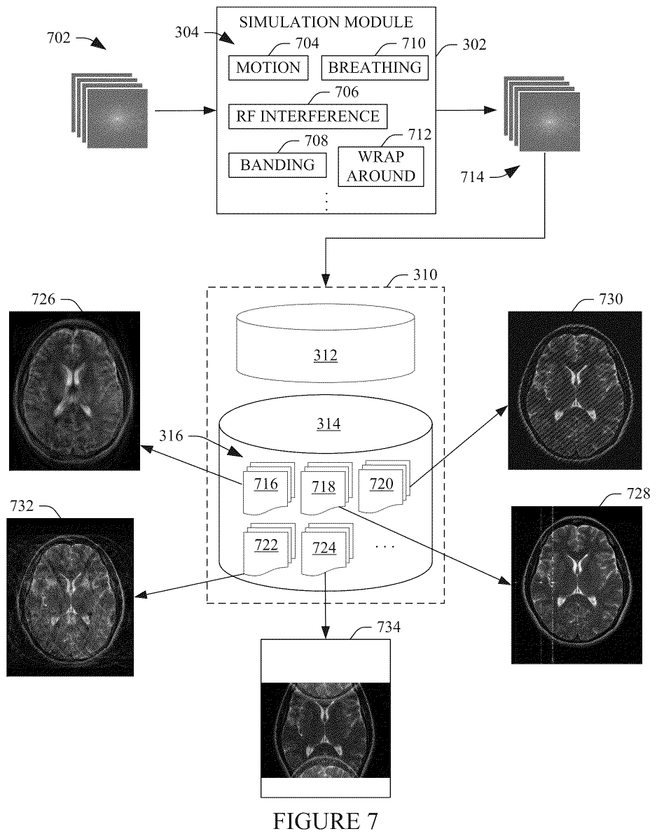

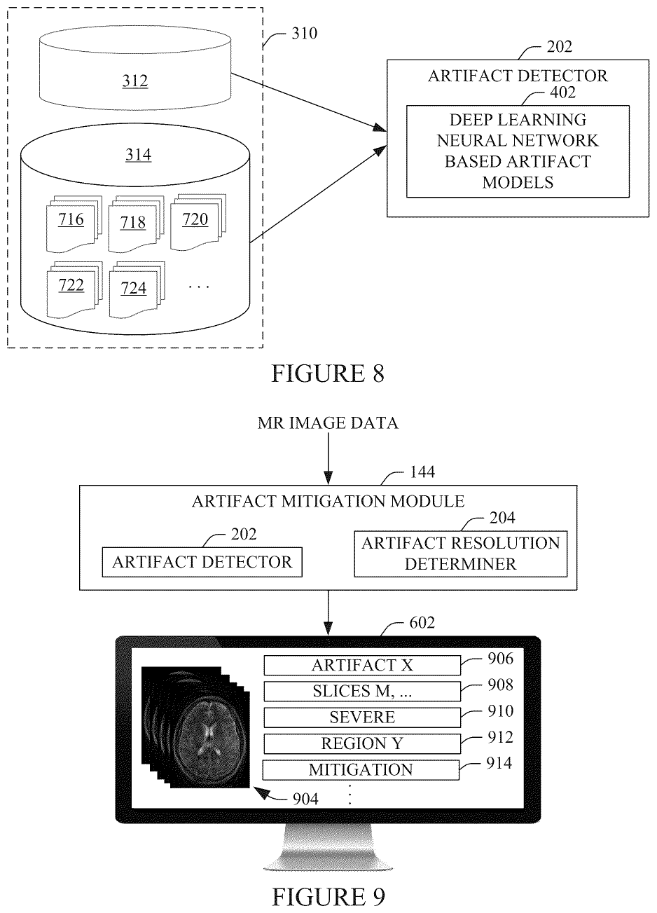

FIGS. 7, 8 and 9 provide an example of training and using the artifact mitigation module 144 in connection with the imaging system 100 (FIG. 1). FIG. 7 schematically illustrates an example of generating MR training data to train the artifact detector 202 (FIG. 2). FIG. 7 schematically illustrates an example of the MR training data. FIG. 8 schematically illustrates an example of the training the artifact detector 202 with the MR training data. FIG. 9 schematically illustrates an example of utilizing the trained artifact detector 202 and the trained artifact resolution determiner 204 to assess raw MR image data and present results and mitigation. FIGS. 7, 8 and 9 are described in connection with MR for explanatory purposes. However, again, the approach described herein is also amenable to other imaging modalities such as CT, PET, SPECT, etc.

Initially referring to FIG. 7, the simulation data generation module 302 receives, as input, artifact free raw image data 702. The simulation module 302 includes the N artifact models 304. In this example, the N artifact models 304 include a motion artifact model 704 (“MOTION”), a radio frequency (RF) interference artifact model 706 (“RF INTERFERENCE”), a banding artifact model 708 (“BANDING”), a breathing artifact model 710 (“BREATHING”), a wrap around artifact model 712 (“WRAP AROUND”) . . . . Again, the N artifact models 304 are configured to introduce artifacts in the raw image data 702 in a physics informed manner to accurately emulate artifact manifestation. Other artifact models can be configured for wrap around, white pixel artifact, etc. The output of the simulation data generation module 302 includes synthetic artifact induced raw image data 714.

The storage unit 310 stores the generated labels in the label storage region 312 and the synthetic artifact induced raw image data 714 stores in the raw image data storage region 314. In the illustrated example, the N sets of synthetic artifact induced raw image data 316 include synthetic motion artifact induced raw image data set 716, synthetic RF interference artifact induced raw image data set 718, synthetic banding artifact induced raw image data set 720, synthetic banding artifact induced raw image data set 722, synthetic banding artifact induced raw image data set 724, . . . . The synthetic artifact induced raw image data 716, 718, 720, 722, 724, . . . respectively correspond to the artifact models 704, 706, 708, 710, 712, . . . .

In this example, the synthetic motion artifact induced raw image data set 716 includes a set of images 726, the synthetic RF interference artifact induced raw image data set 718 includes a set of images 728, the synthetic banding artifact induced raw image data set 720 includes a set of images 730, the synthetic breathing artifact induced raw image data set 722 includes a set of images 732, the synthetic wrap around artifact induced raw image data set 724 includes a set of images 734, . . . . The sets of images 726, 728, 730, 732, 734, . . . respectively correspond to the motion artifact models 704, 706, 708, 710, 712, . . . .

The synthetic artifact induced raw image data sets 726, 728, 730, 732, 734, . . . are delineated by artifact type. In other instance, the synthetic artifact induced raw image data sets 726, 728, 730, 732, 734, . . . are otherwise delineated. For example, in one instance the synthetic artifact induced raw image data sets 726, 728, 730, 732, 734, . . . are delineated into groups such as a patient related artifact group, a hardware related artifact group, an image sequence related artifact group, etc. In another instance, the synthetic artifact induced raw image data sets 726, 728, 730, 732, 734, . . . are delineated into by artifact type and group. In yet another instance, the synthetic artifact induced raw image data sets 726, 728, 730, 732, 734, . . . are otherwise delineated.

An example of a patient related artifact group includes the synthetic motion artifact induced raw image data set 716 and a synthetic breathing artifact induced raw image data set 718. An example of a hardware related artifact group would include the synthetic RF interference artifact induced raw image data set 720 and the synthetic banding artifact induced raw image data set 722. For example, An MR coil that is not properly plugged in may result in white pixel artifact. An example of an image sequence related artifact group would include the synthetic wrap around artifact induced raw image data set 724. For example, a field of view (FOV) that is not large enough may result in wrap around artifact. The label storage region 312 stores corresponding labels for the synthetic banding artifact induced raw images 726, 728, 730, 732, 734,

FIG. 8 schematically illustrates training the artifact detector 202 with the training data created in connection with FIG. 7. Similar to the artifact detector 202 described in connection with FIG. 4, the artifact detector 202 includes complex-valued deep learning neural network based artifact models and/or other AI models. In this example, the artifact detector 202 is trained with the N sets of synthetic artifact induced raw image data 726, 728, 730, 732, 734, . . . and the corresponding labels in the labels 312.

The artifact resolution determiner 204 is trained as described in connection with FIG. 5 and/or otherwise text data, enabling the artifact resolution determiner 204 to perform NLP tasks such as extraction of multi-dimensional information, text summarization, etc.

FIG. 9 schematically illustrates an example of utilizing the trained artifact detector 202 and the artifact resolution determiner 204. The artifact detector 202 receives, as input, raw MR image data generated by an imaging system during a current imaging examination of a patient. The artifact detector 202 processes the received raw MR image data with the complex-valued deep learning neural network and outputs results, e.g., multi-dimensional information about the artifact. The artifact resolution determiner 204 evaluates the results from the artifact detector 202 and presents at least a portion of the results and mitigation.

The summary of the results and mitigation are displayed via the display monitor 602. In this example, the display monitor 602 is a display monitor of the output device 150 of the imaging system 100 (FIG. 1). In this example, the summary includes a set of MR images 904 and indicates the type of the artifact 906 (“ARTIFACT X”) detected in the MR image data, the image slice 908 (“M, . . . ”) that includes the artifact, a severity 910 (“SEVERE”) of the artifact, and the region 912 (“Y, . . . ”) in the slice 908. The mitigation includes mitigation 914. Although this example shows a single artifact (i.e., “ARTIFACT X”) in the summary, it is to be understood that one or more artifacts are detected and the summary summarizes one or more detected artifacts. Again, the imaging technician reviews the results and mitigation and determines any further actions for the current imaging examination.

In one example, the mitigation may include the operator asking the patient to remain as still as they can during the remainder of the imaging examination in response to the artifact mitigation module 144 detecting motion artifact in the raw MR image data. In another example, the mitigation may include the operator ensuring a coil is properly connected in response to the artifact mitigation module 144 detecting white pixel artifact in the raw MR image data. In another example, the mitigation may include the operator increasing a size of the FOV in response to the artifact mitigation module 144 detecting a wrap around artifact in the raw MR image data. In another instance, operator verification of mitigation results in the imaging system automatically implementing the mitigation, e.g., playing a recording that reminds the patient to stay still, etc.

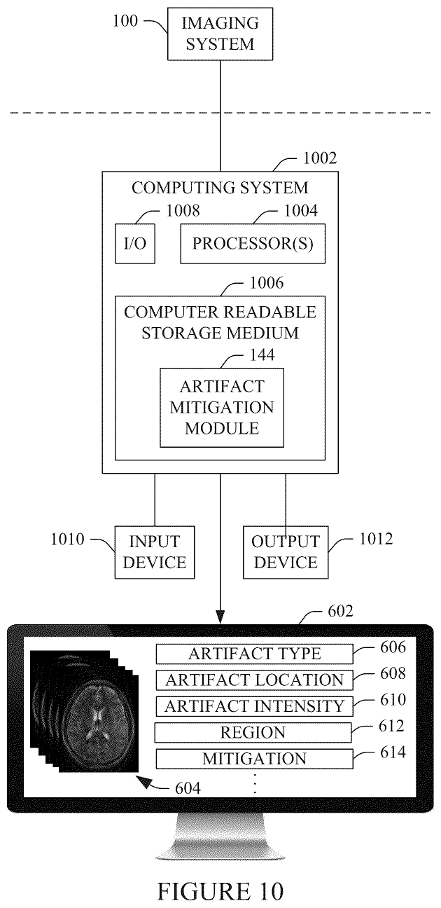

In FIG. 1, the artifact migration module 144 is included in the operator console 138 of the imaging system 100. As briefly discussed above, in other instances the artifact migration module 144 is employed with other medical imaging systems such as CT, PET, SPECT, etc. FIG. 10 includes a variation in which the artifact mitigation module 144 is included in a computing system 1002 that is separate from and not part of any imaging system such as the imaging system 100. Instead, the computing system 1002 is a third party system and/or other system separate from the imaging system 100. In this example, the computing system 1002 is configured to receive raw image data from the imaging system 100 and/or other imaging system.

The computing system 1002 includes a computer, a workstation, etc. The computing system 1002 includes at least one processor 1004 such as a μP, a CPU, a GPU, etc. The computing system 1002 further includes a computer readable storage medium 1006, which includes the artifact mitigation module 144. The at least one processor 1004 is configured to execute instructions of the artifact mitigation module 144 and perform at least the functionality of the artifact mitigation module 144 described herein.

For example, the artifact mitigation module 144 receives, as input, raw image data generated by the imaging system 100 during an imaging examination of a patient. The artifact mitigation module 144 processes the received raw image data with the complex-valued deep learning neural network and generates multi-dimensional information about the artifact. The artifact mitigation module 144 evaluates the multi-dimensional information and presents at least a portion of the multi-dimensional information along with suggested mitigation to avoid and/or reduce the artifact.

The presentation is displayed via a display monitor. The presentation can include a set of images, an artifact type, an indication of which images of the set of images includes the detected artifact, severity of the artifact, an identification of a region within the images that includes the detected artifact, and mitigation, which may include mitigation of patient, imaging system, and/or data acquisition related artifact. The presentation assists the user by providing IQ feedback on the medical images acquired while the patient is still in the scanner, enabling informed decisions regarding scan parameter adjustments, etc. The operator reviews the results and mitigation and determines any further actions for the current imaging examination.

In general, the approach described herein includes AI-based models working on scanner level raw image data to detect artifacts, and based on the detection, AI-based models presents a summarization of the artifact details for the user of the computing system 1002 (or imaging system 100, as discussed above) who decides on a next steps (e.g., further scanning, scan parameter tuning, etc.). The artifact migration module 144 is configured to concurrently detect one or more artifacts during the imaging examination and presents results that provide the user with insight to aid in informed decision-making.

The computing system 1002 further includes input/output (I/O) 1008. An input device 1010 includes a keyboard, mouse, touchscreen, microphone, etc. An output device 1012 includes a human readable device such as a display monitor or the like. The input device 1010 and/or the output device 1012 are in electrical communication with the computing system 1002 through the I/O 1008 and/or otherwise. Similar to the example described in connection with FIG. 6, the summary includes the set of images 604, the artifact type 606, the artifact location 608, the artifact severity 610, the region 612, and the artifact mitigation 614.

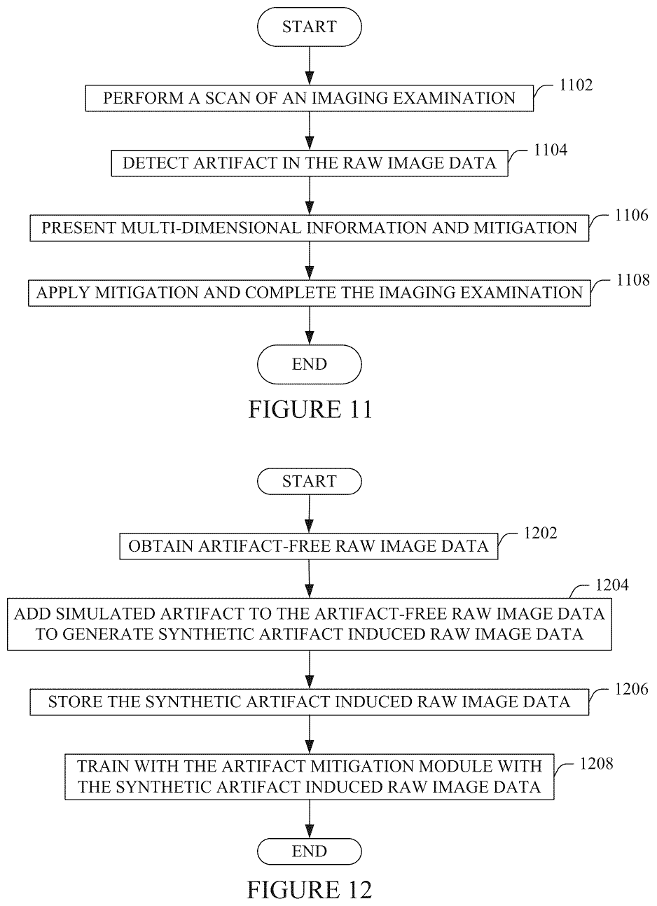

FIG. 11 illustrates a non-limiting example of a flow chart for a computer-implemented method for assessing raw medical image data for artifact during an imaging examination to mitigating image artifact before completion of the imaging examination. It is to be appreciated that the ordering of the acts in the method is not limiting. As such, other orderings are contemplated herein. In addition, one or more acts may be omitted, and/or one or more additional acts may be included.

At 1102, a patient is scanned during an imaging examination with a medical imaging system, generating raw image data, as described herein and/or otherwise. For example, the raw image data can be MR, CT, PET, SPECT, etc. raw image data. The raw image data is provided to the trained artifact mitigation module 144, as described herein and/or otherwise. At 1104, the trained artifact detector 202 processes the raw image data, detecting artifact in the raw image data and multi-dimensional information about the artifact, as described herein and/or otherwise. In one instance, the artifact detector 202 includes complex-valued deep learning neural network artifact models that are trained to detect artifact in complex-valued data such as the raw image data from a medical imaging system.

At 1106, the trained artifact resolution determiner 204 processes the multi-dimensional information and presents at least a portion of the multi-dimensional information along with suggested mitigation, as described herein and/or otherwise. In one instance, the artifact resolution determiner 204 includes large vision-language models trained to extract the multi-dimensional information and generate a summary of the multi-dimensional information. For example, in one instance the artifact resolution determiner 204 presents images, a type of artifact, an indication of which slices include the artifact, a severity of the artifact, an indication of a region in a slice that includes the artifact, etc.

At 1108, the mitigation is applied and the imaging examination is completed, as described herein and/or otherwise. Examples of mitigation in connection with raw MR image data includes the following: the operator may ask the patient to remain as still as they can during the remainder of the imaging examination to avoid and/or reduce motion artifact; the operator may secure a plug connection of a coil to avoid and/or reduce white pixel artifact; the operator may increase a size of the FOV to avoid and/or reduce wrap around artifact, etc.

In general, the raw image data generated during the imaging examination after the mitigation includes less artifact than the raw image data generated during the imaging examination before the assessment and the mitigation. As such, the approached described assists the operator of the imaging system by providing IQ feedback on the medical images acquired while the patient is still in the scanner, enabling informed decisions regarding scan parameter adjustments, etc. before the imaging examination is complete.

FIG. 12 illustrates a non-limiting example of a flow chart for a computer-implemented method for training the artifact mitigation module 144 with artifact free raw image data. It is to be appreciated that the ordering of the acts in the method is not limiting. As such, other orderings are contemplated herein. In addition, one or more acts may be omitted, and/or one or more additional acts may be included.

At 1202, artifact free raw image data is obtained, as described herein and/or otherwise. At 1204, the simulation data generation module 302 adds artifact to the artifact free raw image data, generating synthetic artifact induced raw image data, as described herein and/or otherwise. For example, in one instance the simulation data generation module 302 includes the artifact models 304 that are configured to introduce artifacts in the raw image data in a physics informed manner to accurately emulate artifact manifestation, to replicate artifacts in both the magnitude and phase data of the raw image data. In one instance, the simulation includes simulating an artifact and/or a combination of artifacts.

At 1206, the synthetic artifact induced raw image data and corresponding labels are stored in the memory 142, as described herein and/or otherwise. At 1208, the artifact detector 202 is trained to detect artifact based on the synthetic artifact induced raw image data, as described herein and/or otherwise. As discussed herein, in one instance this includes training complex-valued deep learning based artifact models for concurrently detecting and classifying multiple different artifacts.

The above method(s) can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A computer-implemented method, comprising:
obtaining raw image data from a medical imaging system during an imaging examination performed by the medical imaging system;
processing the raw image data with trained artificial intelligence deep learning based artifact models to detect artifact from the raw medical image data and generate multi-dimensional information about the detected artifact;
processing at least the multi-dimensional information with trained large vision-language models to generate a summary of the detected artifact and determine mitigation for the artifact; and
presenting the summary and mitigation, wherein the summary includes a set of images and indicates a type of each detected artifact, an identification of images in the set of images that includes the detected artifact, and a severity of the detected artifact, and the mitigation provides acts for reducing the image artifact for the remainder of the imaging examination, wherein a least a portion of the mitigation is implemented.

2. The computer-implemented method of claim 1, further comprising:
obtaining artifact-free raw image data;
simulating at least one of artifact and a combination of artifacts; and
adding the simulated artifact to the artifact-free raw image data to generate synthetic artifact induced raw image data.

3. The computer-implemented method of claim 2, wherein the artifact-free raw image data corresponds to images that were previously evaluated for artifact and labeled and determined to be of diagnostic quality.

4. The computer-implemented method of claim 2, further comprising:
simulating the artifact in a physics informed manner to accurately emulate artifact manifestation during image acquisition.

5. The computer-implemented method of claim 2, further comprising:
processing the raw image data with a processor of an operator console of the medical imaging system.

6. The computer-implemented method of claim 5, further comprising:
adjusting an aspect of the imaging examination based on the mitigation and completing the imaging examination.

7. The computer-implemented method of claim 6, wherein a first set of images generated before the mitigation include a first level of an artifact, a second set of images generated after the mitigation include a second level of the artifact, wherein the second level is lower than the first level.

8. The computer-implemented method of claim 1, wherein the raw image data includes k-space data.

9. The computer-implemented method of claim 1, wherein the raw image data includes sinogram data.

10. A system, comprising:
a memory including an artifact mitigation module configured to assess raw medical image data for artifact and provide a summary of the assessment and mitigation to reduce the artifact,
wherein the artifact mitigation module includes:
deep learning based artifact models trained to assess raw image data, detect artifact in the raw medical image data, and generate multi-dimensional information about the detected artifact, and
large vision-language models trained to process the multi-dimensional information and generate a summary of the detected artifact and mitigation for the artifact;
a processor configured to:
execute the trained deep learning based artifact models to assess raw image data received from an imaging system, detect artifact in the raw medical image data, and generate multi-dimensional information about the detected artifact,
execute the trained large vision-language models trained to process the multi-dimensional information and generate a summary of the detected artifact and mitigation for the artifact; and
a display configured to present the summary of the detected artifact and the mitigation for the artifact.

11. The system of claim 10, wherein the raw image data includes k-space data.

12. The system of claim 10, wherein the raw image data includes sinogram data.

13. The system of claim 10, wherein the mitigation reduces the artifact for a remainder of the imaging examination.

14. A computer readable storage medium encoded with computer executable instructions, which when executed by a processor, causes the processor to:
obtain raw image data from a medical imaging system during an imaging examination performed by the medical imaging system;
process the raw image data with trained artificial intelligence deep learning based artifact models to detect artifact from the raw medical image data and generate multi-dimensional information about the detected artifact;
process the multi-dimensional information with train large vision-language models to generate a summary of the detected artifact and determine mitigation for the artifact; and
present the summary and mitigation, wherein the summary includes a set of images and indicates a type of each detected artifact, an identification of images in the set of images that includes the detected artifact, and a severity of the detected artifact, and the mitigation provides acts for reducing the image artifact for the remainder of the imaging examination.

15. The computer readable storage medium of claim 14, wherein the instructions further cause the processor to:
obtain artifact-free raw image data;
simulate at least on of artifact and a combination of artifacts; and
add the simulated artifact to the artifact-free raw image data to generate synthetic artifact induced raw image data.

16. The computer readable storage medium of claim 15, wherein the artifact-free raw image data corresponds to images that were previously evaluated for artifact and labeled and determined to be of diagnostic quality.

17. The computer readable storage medium of claim 15, wherein the instructions further cause the processor to:
simulate the artifact in a physics informed manner to accurately emulate artifact manifestation during image acquisition.

18. The computer readable storage medium of claim 15, wherein the instructions further cause the processor to:
process the raw image data with a processor of an operator console of the medical imaging system.

19. The computer readable storage medium of claim 18, wherein the instructions further cause the processor to:
adjust an aspect of the imaging examination based on the mitigation and completing the imaging examination.

20. The computer readable storage medium of claim 19, wherein a first set of images generated before the mitigation include a first level of an artifact, a second set of images generated after the mitigation include a second level of the artifact, wherein the second level is lower than the first level.
